(19) United States
(12) Patent Application Publication
(10) Pub. No.: US 2010/0234864 A1
(43) Pub. Date: Sep. 16, 2010

(54) Three-Dimensional Cutting Instrument

(75) Inventor: Christopher Guild Keller, El Cerrito, CA (US)

Correspondence Address: FENWICK & WEST LLP, SILICON VALLEY CENTER, 801 CALIFORNIA STREET, MOUNTAIN VIEW, CA, 94041, US

(73) Assignee: Mynosys Cellular Devices, Inc., Albany, CA (US)

(21) Appl. No.: 12/377,231

(22) PCT Filed: Aug. 13, 2007
(86) PCT No.: PCT/US07/75836

§ 371 Date: May 28, 2010

Related U.S. Application Data
(60) Provisional application No. 60/837,401, filed on Aug. 11, 2006.

(51) Int. Cl. A61B 17/22

(52) U.S. Cl. 606/159

(57) ABSTRACT

A cutting instrument includes a blade and a cutting edge that is curved in a direction having a vector component that is transverse to a cutting direction of the instrument, thereby forming a three-dimensional cutting edge. In use, this structure allows the cutting instrument to be drawn across tissue, without necessarily rotating, and separate a strip from the tissue. Various geometries for the cutting instrument can be created by forming a planar blade, heating the blade, and then plastically deforming the blade around a mandrel to achieve the desired three-dimensional geometry.

### CROSS REFERENCE TO RELATED APPLICATIONS

This application claims the benefit of U.S. Provisional Application No. 60/837,401, filed Aug. 11, 2006, which is incorporated by reference in its entirety.

### BACKGROUND

This invention relates generally to cutting instruments for various applications, including microsurgery, and in particular to making and using cutting instruments that have a three-dimensional cutting blade.

Conventional knives made from metal or those having diamond-tipped edges are relatively large for microsurgical applications; that is, they have cutting edges that are considerably large when viewed on the atomic scale. Typically, such knives have cutting edges with a radius of curvature ranging from 500 to 1000 Angstroms, or higher. Because of the large size of their cutting edge, these knives provide poor surgical precision and cause unnecessary and undesirable destruction of tissue at the cellular level.

To address this size deficiency of the large prior art knives, microsurgical cutting instruments have been made from single crystal silicon. It has been found that etching of silicon can produce near-atomically sharp cutting edges (e.g., radius of curvature about 10 Angstroms), resulting in microknives that are appropriate for certain microsurgical applications. But these microknives also have their limitations. Because they are made by etching silicon crystals, the microknives are all straight and planar. This planar geometry allows the microknives to make precise incisions; however, more complicated cuts such as the removal of a strip of tissue would require multiple passes of the knife. Given the small scale of microsurgery, it can be difficult to align the precise cuts to be made with each pass of the microknife to achieve the desired removal of a strip of tissue.

Accordingly, it may often be desirable to cut out a strip of tissue with a single pass of a knife blade, leaving a groove in the tissue where the strip of tissue was removed. Examples of applications where it may be desirable to remove a strip rather than make a single incision include biopsies and microsurgeries to remove undesirable cells or deposits (i.e., plaques). This is not feasible with existing flat cutting instruments.

### SUMMARY

Embodiments of the invention provide microsurgical cutting instruments that are curved in such a way that when the instrument is drawn across a confronting tissue, it will cut a strip from the tissue with a single pass of the instrument. The strip of tissue cut may be completely removed from the surrounding tissue or it may be left attached by an end of the strip. The three-dimensional microsurgical cutting instrument may be formed in various geometries, including U-shaped, helical, and mirrored-helical.

One embodiment of the microknife comprises a blade formed from silicon. The blade includes at least one cutting edge, which preferably has a radius of curvature of less than about 50 Angstroms, and which defines a cutting direction of the microknife. The cutting edge and blade are curved out of the plane of the as-etched planar blade and in a direction having a vector component transverse to the cutting direction, thereby forming a three-dimensional microsurgical cutting instrument that can be used to remove a strip of tissue. During use of the microsurgical cutting instrument, in one embodiment, the blade is is advanced towards and into tissue, and a strip of tissue is separated by the cutting action of the blade.

In one embodiment, a method for making a three-dimensional microsurgical cutting instrument comprises etching a planar microknife in silicon. The planar microknife is then heated and plastically deformed against a curved mandrel, yielding a microknife with a three-dimensionally curved blade. The microknife may be mounted in a handle structure that is suitable for the instrument's intended application (e.g., attached to a rod, placed within a catheter, etc.). The blade may be mounted to the handle structure so that the blade approaches the tissue at a shallow angle. By cutting with the blade at a shallow angle, the cutting action is primarily slicing rather than chopping, which reduces the drag force on the microknife.

In various embodiments described herein, the knives can be used for performing microsurgical procedures. Beneficially, the microknives may cut with a lateral force only, so that no rotation of the curved knives is required. This may be important for microsurgery because much greater forces can be transmitted by micromechanical beams in axial compression or axial tension than by torsion through axial rotation. In addition, embodiments of the curved microknives described herein have many other useful applications for cutting materials other than biological tissues.

### BRIEF DESCRIPTION OF THE DRAWINGS

FIGS. 1A and 1B illustrate straight and chevron geometries, respectively, for a planar blade for a microsurgical cutting instrument, in accordance with an embodiment of the invention.

FIGS. 2A through 2C illustrate three-dimensional geometries for the microsurgical cutting instrument, in accordance with an embodiment of the invention.

FIGS. 3A through 3D are axial views of different geometries for a helical microsurgical cutting instrument, and FIG. 3E is a perspective view of the helical blade in FIG. 3D, in accordance with an embodiment of the invention.

FIGS. 4A through 4D illustrate a process for forming a three-dimensional microknife from a planar microknife, in accordance with an embodiment of the invention.

FIGS. 5A and 5B illustrate a process for forming a helical microknife, in accordance with an embodiment of the invention.

FIGS. 6A and 6B illustrate a process for forming a mirrored helical microknife, in is accordance with an embodiment of the invention.

FIGS. 7, 8, and 9 illustrate a use of a microsurgical cutting instrument having a U-shape, a helical shape, and a mirrored helical shape, respectively, in accordance with an embodiment of the invention.

FIGS. 10A and 10B illustrate an angular relationship between a helical cutting instrument and a tissue surface to be cut, in accordance with an embodiment of the invention.

FIG. 11A is a side view of a path followed by a cutting edge of a blade, and FIG. 11B shows the resulting groove and piece of tissue removed, in accordance with an embodiment of the invention.

FIG. 12 shows a strip of tissue that is left attached at one end after a cut, in accordance with an embodiment of the invention.

FIGS. 13A and 13B show, respectively, perspective and axial views of tissue cut by a J-shaped blade so that the strip remains attached to the tissue along one side of its length, in accordance with an embodiment of the invention.

FIGS. 14 through 18 show details of mandrels used to form various blade geometries, in accordance with an embodiment of the invention.

FIGS. 19 and 20 show a blade having a conical helix geometry, in accordance with an embodiment of the invention.

FIG. 21 shows a knife having two blades, including a left-handed helix and a right-handed helix, in accordance with an embodiment of the invention.

FIGS. 22A through 22D show different cross sections of a microknife obtainable through wet etching, in accordance with embodiments of the invention.

FIGS. 23A and B show a knife blade defined by wet etching on a compliant flexure mechanism defined by deep reactive ion etching, and FIG. 24 shows the deflection of the knife in a direction different than the curvature of the knife, in accordance with an embodiment of the invention.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

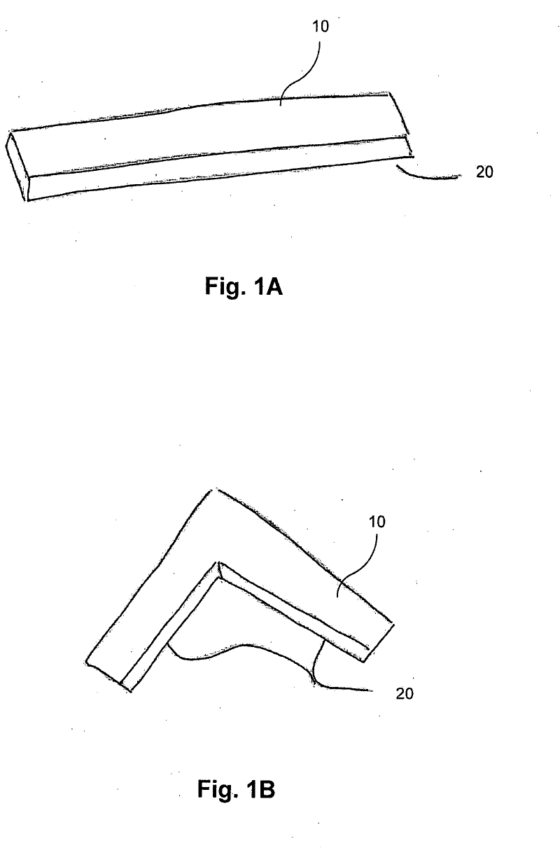

Embodiments of the cutting, instruments described herein are formed from microknives having various structures, geometries, and materials. For example, the microknives may be etched from silicon or other covalently bonded materials, or made from various, glasses. Silicon microsurgical cutting instruments can be conveniently etched in various planar geometries. FIGS. 1A and 1B illustrate a straight geometry and a chevron geometry, respectively, for the planar microknife. In each of these geometries, the planar microknife includes a blade section 10 and at least one cutting edge 20 of the blade 10. Depending on the desired application, various other planar geometries may be used for the microknife. The radius of curvature of the cutting edge 20 of blade 10 of the microknife is less than or equal to about 50 Angstroms.

Various methods for forming microsurgical cutting instruments that can be used with embodiments of the invention, including instruments having self-sharpening cutting edges, are disclosed in International Application No. PCT/US07/61701, filed Feb. 6, 2007, which is incorporated by reference in its entirety.

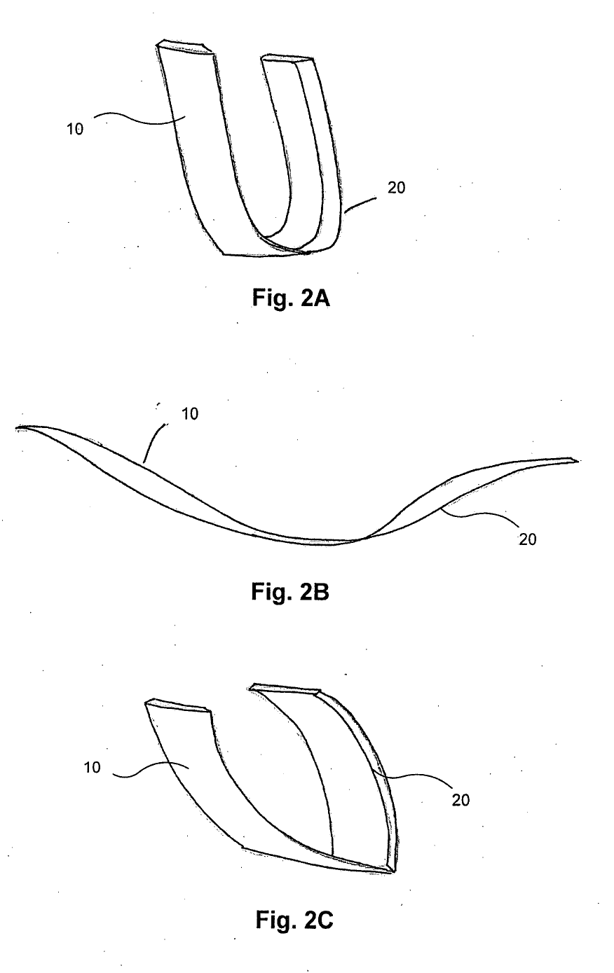

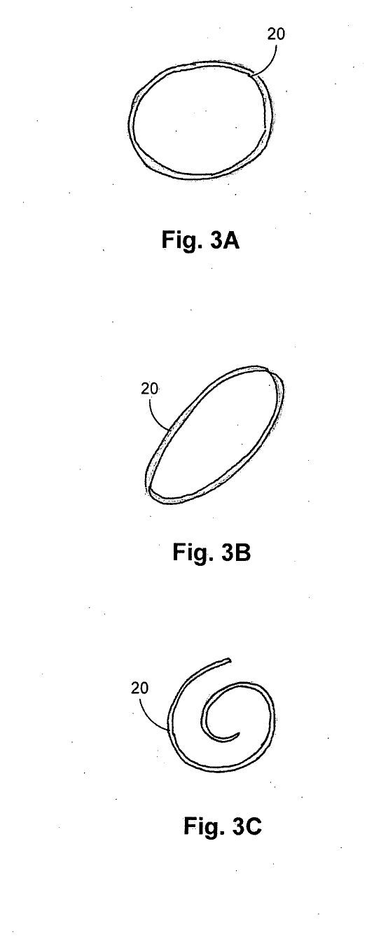

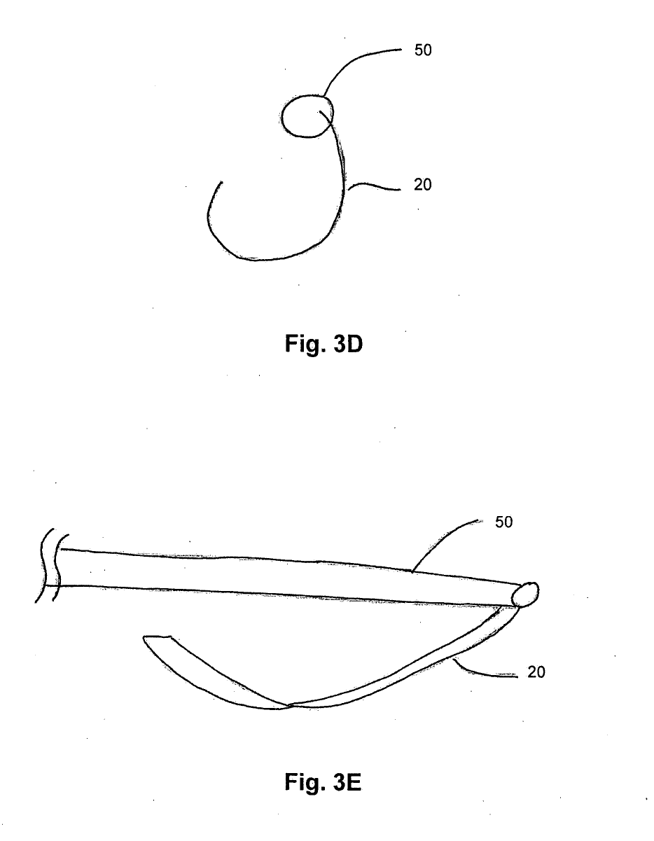

To form the desired three-dimensional microsurgical cutting instrument, the planar microknife can be bent into a third dimension to yield a curved shape. FIGS. 2A through 2C illustrate a few of the possible three-dimensional geometries that can be achieved. For example, the microknife can be curved into a U-shape, as shown in FIG. 2A; a helix, as shown in FIG. 2B; or a mirrored helix, as shown in FIG. 2C. The U-shaped and helical microknives may be formed, e.g., from a straight planar geometry, such as that shown in FIG. 1A, and the mirrored helix may be formed from the chevron planar geometry, such as that shown in FIG. 1B. Moreover, the helix shape shown in FIG. 2B may be bent in various curvatures to achieve different geometries as viewed in an axial direction. For example, FIG. 3A is an axial view of a microknife formed in a circular helix, FIG. 3B shows the knife formed in an elliptical, and FIG. 3C shows the knife form in a conical helix. FIG. 3D shows a helical knife that is attached to a handle 50 at only one end and curved so that its cutting edge 20 is J-shaped in the axial view.

As described, the silicon microknife after being etched has a planar geometry. To form a curved geometry as described above from the planar silicon blade, a plastic deformation process under application of heat and stress may be used. At high temperatures, the planar silicon knife structure can be bent to form various curved shaped, such as those shown in FIGS. 2A through 2C.

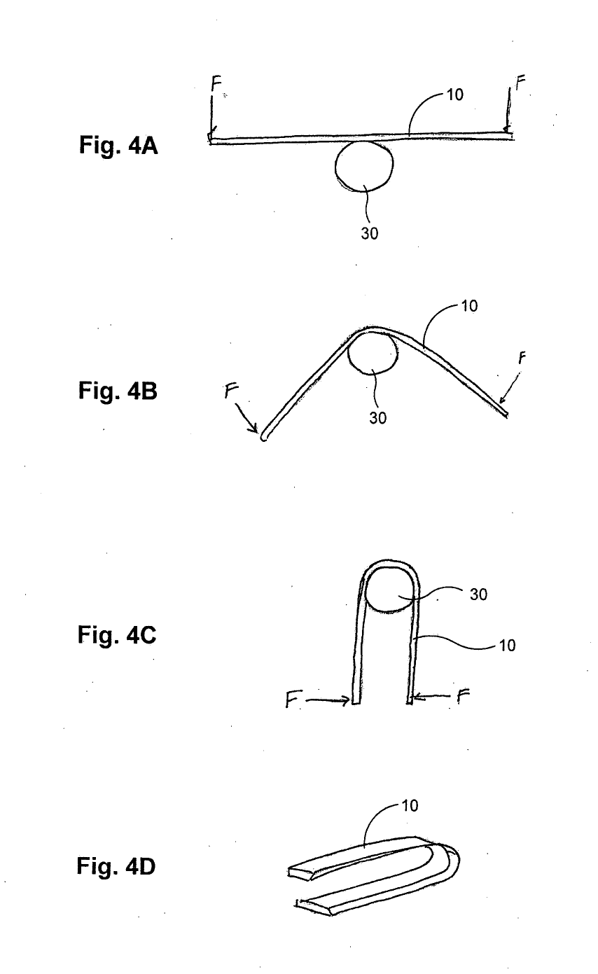

FIGS. 4A through 4D illustrate one embodiment of a process for forming a three-dimensional geometry for the microknife. In a first step, shown in FIG. 4A, a planar microsurgical cutting instrument blade 10 is set against a mandrel 30, where the midpoint of the blade 10 touches the surface of a curved mandrel 30. In one embodiment, the mandrel 30 comprises a fused silica tube with a nichrome wire inside the tube. This fused silica tube can be heated by passing an electrical current through the wire, thereby raising the temperature of the outer surface of the fused silica tube to a high temperature (e.g., typically 900° C., but may be within a range from about 850° C. to about 1300° C.). Other materials stable at high temperature can be used instead of fused silica, such as silicon carbide. In the case of doped silicon carbide having suitable electrical conductivity, heating can be achieved by passing the current directly through the mandrel 30 instead of a nichrome wire. In alternative embodiments, the blade 10 and mandrel 30 may be heated using other heating mechanisms, such as a gas flame. Preferably, the process is performed in an inert atmosphere (e.g., Ar or N₂) to avoid dulling the cutting edge 20 of the blade 10.

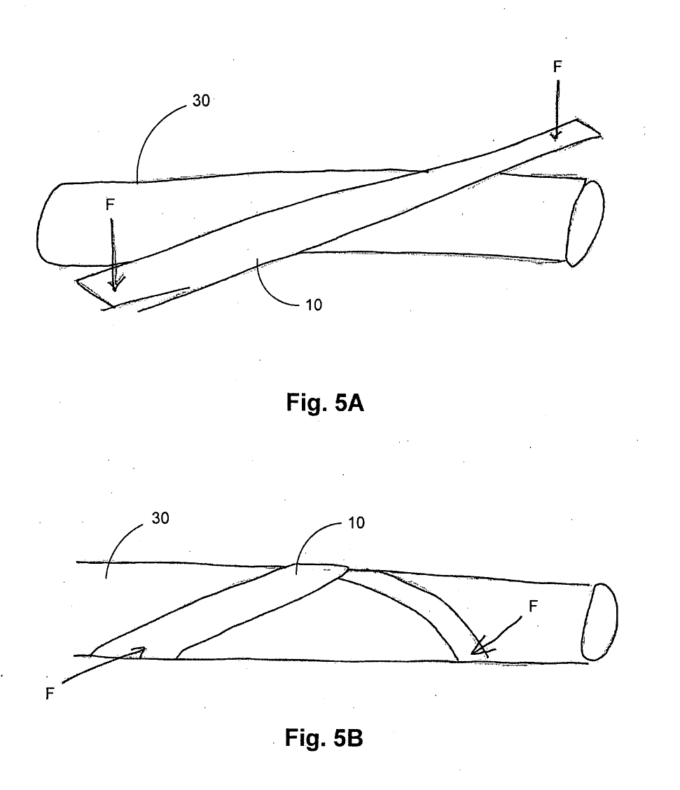

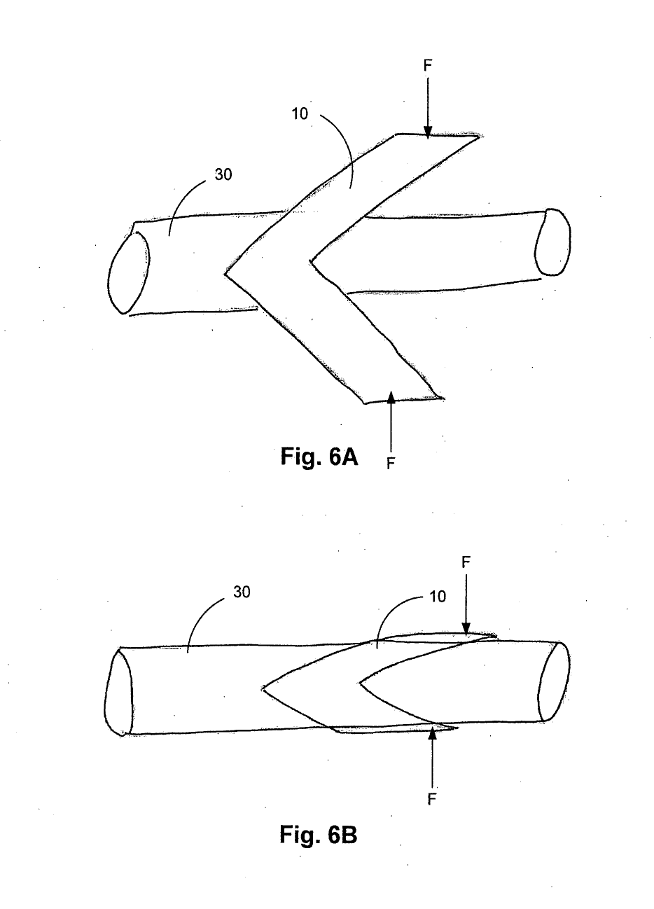

At temperatures of about 900° C. or greater, silicon can be plastically deformed if stress is applied. Accordingly, a force F is applied to opposite ends of the blade 10, which causes the blade 10 to bend around the mandrel 30, as shown in FIGS. 4B and 4C. The result of this process is a U-shaped blade 10, as shown in FIG. 4D. It can be appreciated that various other configuration of forcing the blade 10 around a mandrel 30 can yield other three-dimensional geometries. For example, FIGS. 5A and 5B illustrate a helical geometry being formed by wrapping a straight blade 30 around a cylindrical mandrel 30. Helical geometries with elliptical or conical cross sections (as shown in FIGS. 3B and 3C, respectively) may be formed by using elliptical or conical mandrel 30, respectively. Similarly, FIGS. 6A and 6B illustrate a mirrored helical geometry being formed by bending a chevron-shaped blade 10 around a cylindrical mandrel 30.

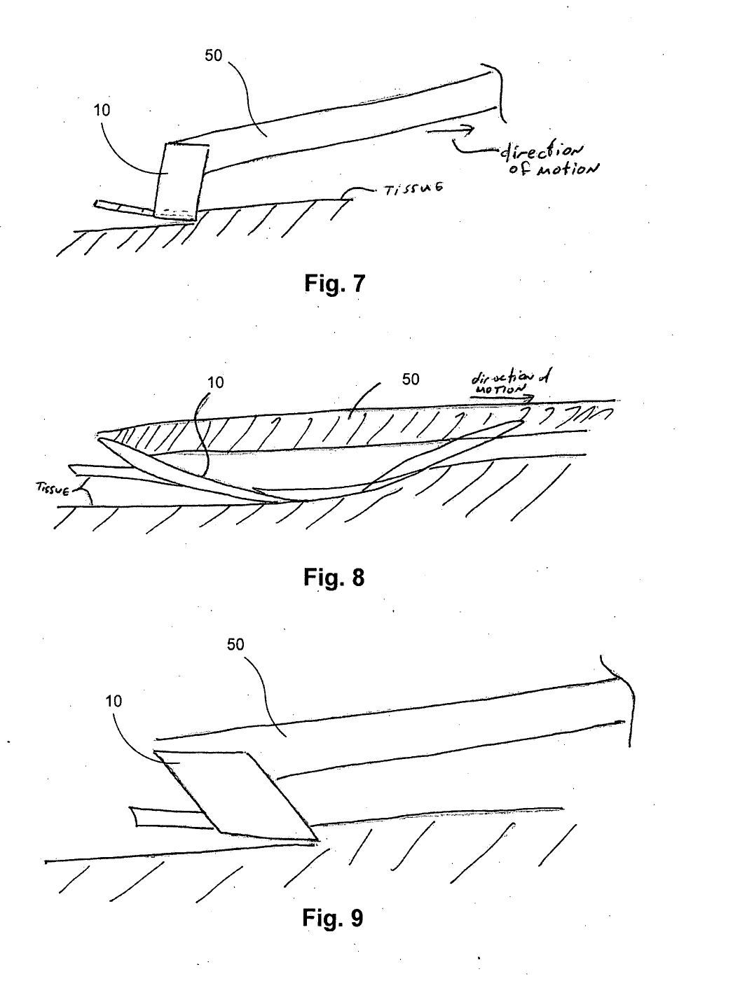

As illustrated in the example embodiments of FIGS. 7-9, the three-dimensional microknife may be attached to a handle structure 50 that is suitable for the intended application of the microknife. For example, the microknife may be attached to a handle for cutting by hand or along a guide for more precise cutting. In one embodiment, the blade and handle structure may be placed within a catheter for insertion through a lumen structure, and then the blade can be exposed (e.g., by pulling back the catheter relative to the blade or moving the blade outside of the catheter) to expose the blade for cutting (e.g., by moving the instrument together with the catheter). Where the microknife is helical, the blade may be mounted around a mandrel having a cylindrical or prismatic structure, which may be pulled across tissue to produce a desired cut.

FIGS. 7, 8, and 9 illustrate the use of a microsurgical cutting instrument having a U-shape, a helical shape, and a mirrored helical shape, respectively. To cut with the instrument, an operator advances the cutting blade of the instrument towards and into tissue to be cut. The result is a strip that is removed from the tissue. The strip may be completely removed by continuing the cut through and out of the tissue. This type of cut may be useful for removing a volume of tissue, such as for a biopsy. The cross sectional shape, width, and height of the cut and of the removed tissue are determined by the geometry of the axial projection of the blade. Alternatively, the strip may be left attached to the tissue at one end of the strip, by stopping the advancement of the instrument before it cuts through and out of the tissue. This type of cut may be useful when the tissue is being cut away not to remove the tissue but to expose something behind the strip of tissue. Leaving the strip of tissue attached may allow the strip to be replaced once a surgical procedure is completed so it can heal with the surrounding tissue.

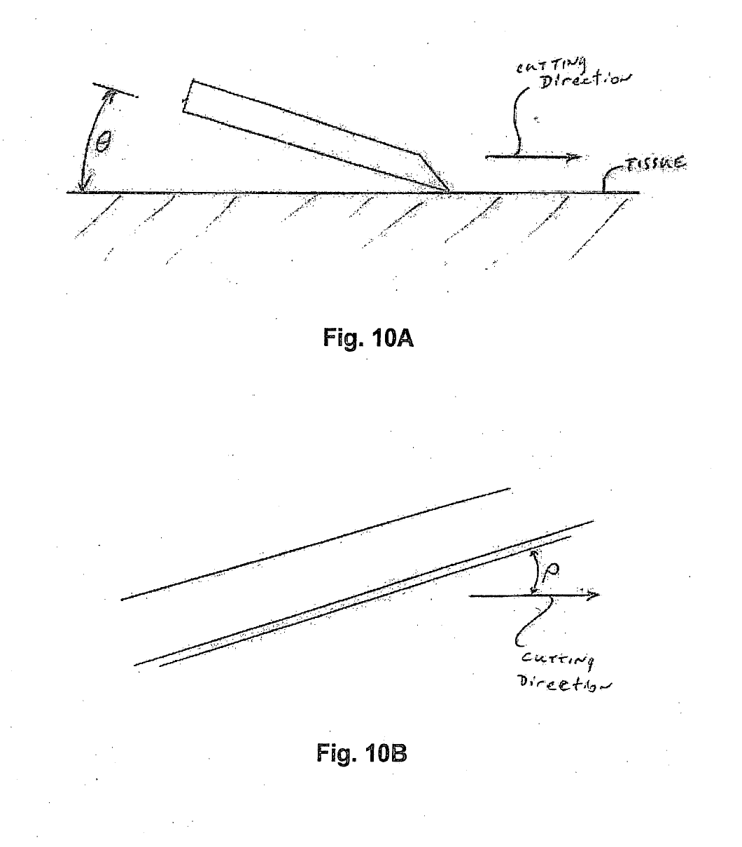

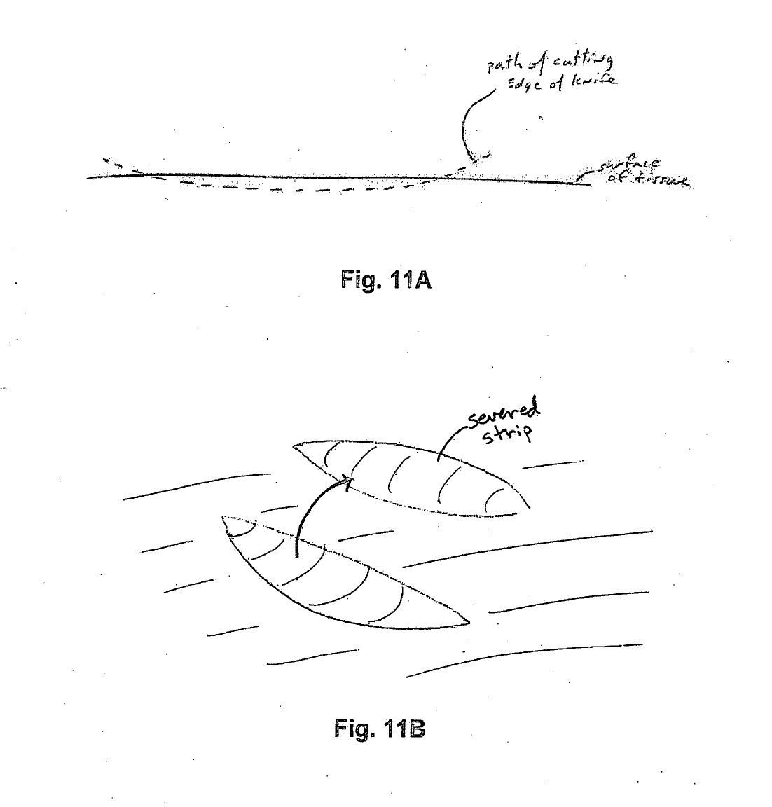

FIGS. 10A and 10B show parameters of orientation of the blade with respect to the surface of the tissue being cut. FIG. 10A illustrates a relief angle θ. If θ were zero, the whole side of the blade would be pressing against the surface of the tissue, which would make it more difficult for the cutting edge to dig into the tissue and start slicing. The value selected for the relief angle θ may depend on the mechanical properties of the particular tissue to be addressed. For typical tissue, θ will be in the range of 5 to 15 degrees. FIG. 10 B shows the pitch angle ρ. This is also the angle between the cutting edge and the direction of motion of the blade. The smaller the value of ρ, the longer the helix must be for one period. Also, the smaller the value of ρ, the lower the required cutting force will be. Typically, ρ will be within the range of 5 to 45 degrees.

The amount of force needed to cut through tissue depends, in part, on the angle at which the blade is advanced through the tissue. The U-shaped blade shown in FIG. 7 will require the highest cutting force because it has to be pushed through the tissue at the least advantageous angle. The mirrored helix design shown in FIG. 9 requires less cutting force because it is approaching the tissue at an angle of about 45 degrees. However, the lowest cutting force can be achieved with the helix design shown in FIG. 8 because the angle can be made arbitrarily low. In the helix design, as the angle between the blade and tissue is decreased, the length of the blade increases. The causes the length of the minimum cut to increase.

FIG. 11A illustrates a path followed by a point on the cutting edge of a blade as it moves toward and into the tissue, slices through it, and finally moves up and out of the tissue. FIG. 11B shows the resulting groove and severed strip of tissue. The cross sectional shape of the cut is determined by the axial projection of the blade. The width, length, and depth of cut are controllable by the surgeon, within the limits of the size of the blade.

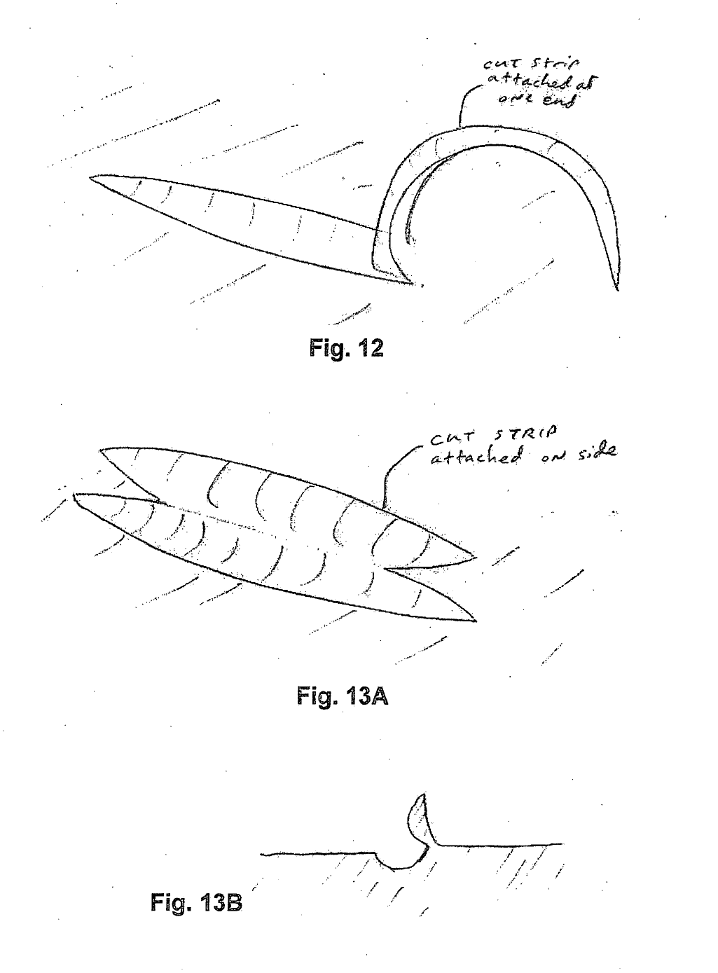

FIG. 12 shows the case of a strip of tissue intentionally left attached to the main body of tissue at one end. This kind of cut may be performed by stopping the knife and moving it in the reverse direction until it is free of the cut strip.

FIGS. 13A and 13B show perspective and axial views, respectively, of a strip cut by a blade attached at only one end to a handle such that the axial projection (i.e., in the cutting direction) of the blade is J-shaped. Tissue along one side of the length of the strip remains uncut and serves as a hinge for rotation of the strip out of the groove, and then possibly back into the groove.

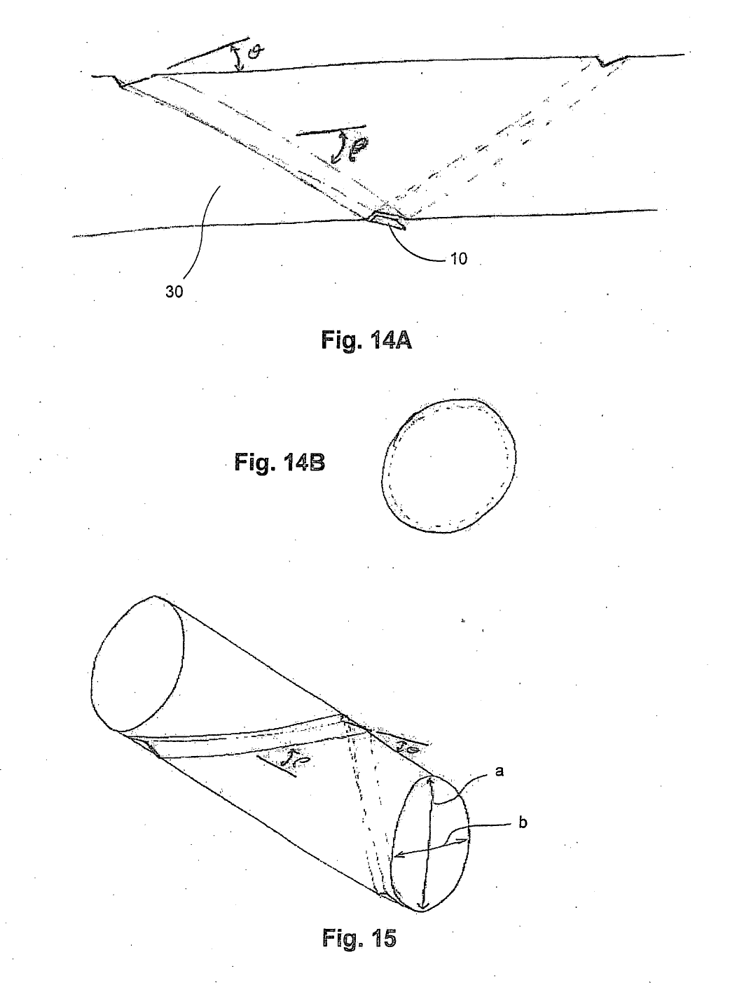

FIGS. 14 through 18 show various embodiments of a mandrel for forming blades into the desired three-dimensional curves. In each case a groove has been ground into the mandrel that sets the relief angle and the pitch angle of the blade. In one embodiment, one end of a blade is first placed in the groove, and then the other end of the blade is bent into it under heated conditions. In other embodiments, the relief angle may be set by twisting the blade when it is mounted in the handle.

FIG. 14A shows a side view of a mandrel to form a blade that is a circular helix having a relief angle θ, and a pitch angle ρ. FIG. 14 B is an axial view of this mandrel.

FIG. 15 is a perspective view of a mandrel that can be used for forming a blade that is an elliptical helix having a relief angle θ, a pitch angle ρ, a major axis a, and a minor axis b.

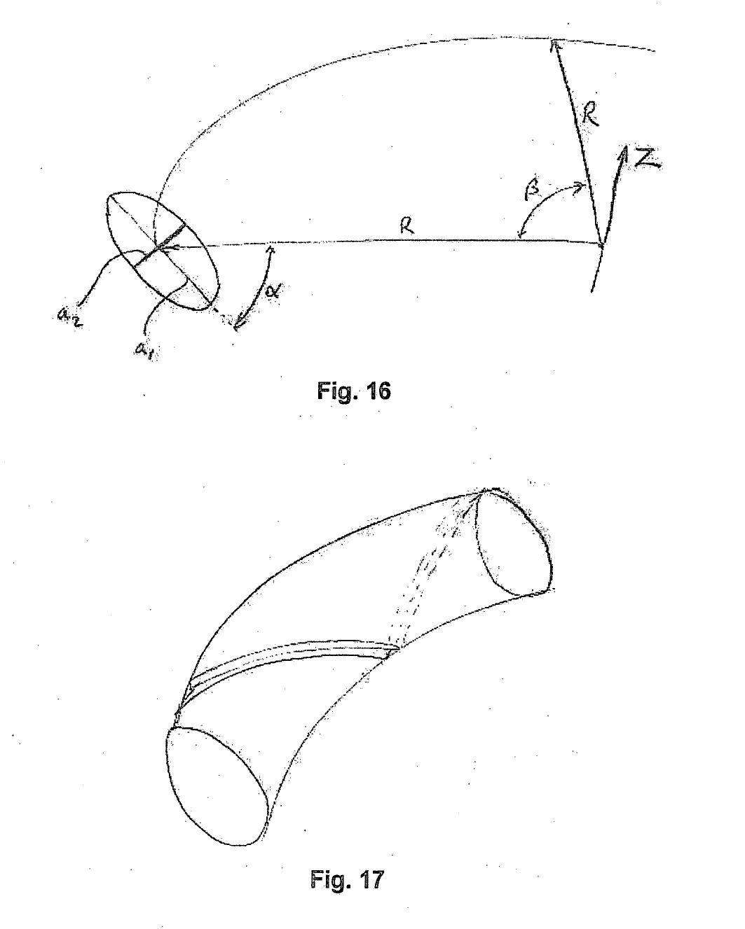

FIG. 16 shows the geometric parameters for a mandrel that is a segment of an elliptical torus. The ellipse that is the generator of the torus has a major axis a₁ that is inclined to the plane of the torus by an angle α, and has minor axis a₂. The torus is generated by revolving the generator ellipse about axis Z (which is perpendicular to the plane of the torus) at a radius R through an angle β. FIG. 17 illustrates the resulting mandrel.

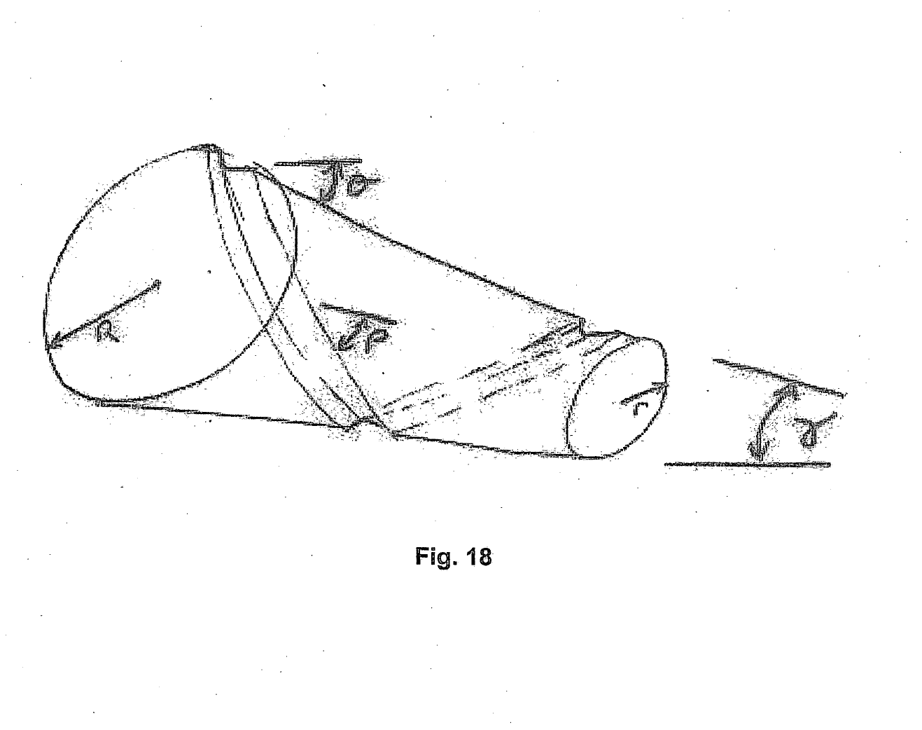

FIG. 18 shows a mandrel having a radius r at one end and a larger radius R at the other end, where the mandrel is for forming a blade that is a right circular conical helix having a relief angle θ, a pitch angle ρ, and a cone angle γ.

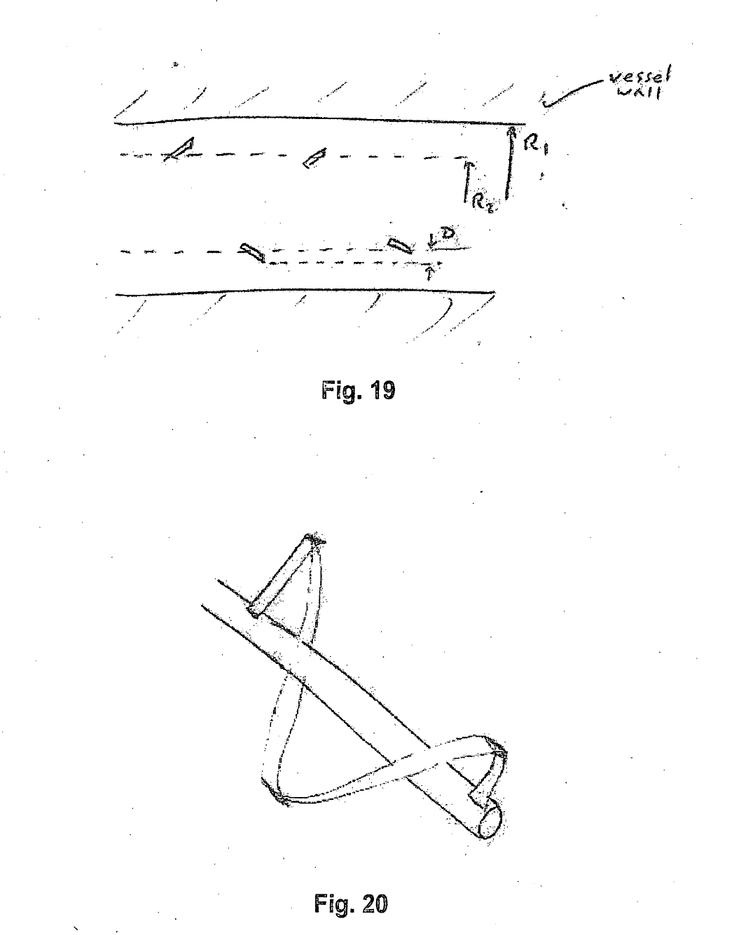

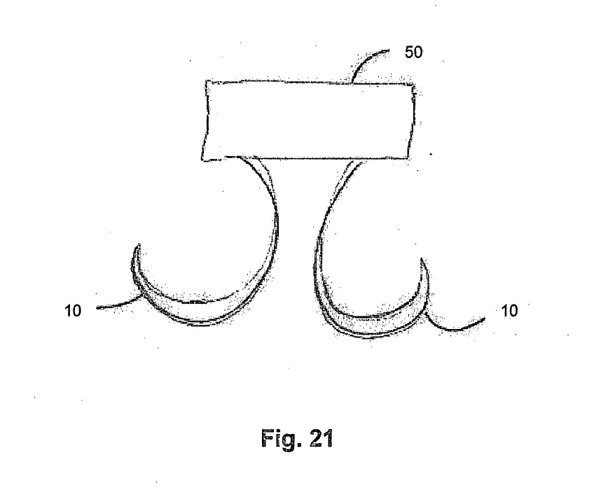

FIG. 19 shows in longitudinal cross section a cylindrical vessel of circular transverse cross section having a radius R₁; which has a build up of deposits leaving a lumen of R₂. This could be an artery that has been building up fatty deposits. Conventionally, such arteries are cleared by rotating grinding tools mounted on catheters. These tools generate many debris particles that may later get lodged in capillaries. In contrast, a conical helix blade of the present invention may be pushed without rotation through the lumen of the deposit, and each successive period of the conical helix of the blade cuts deeper in the deposit by a distance D. The cut material is retained on the tool and removed from the artery with the tool. FIG. 20 is a perspective view of the blade of FIG. 19, which might be used for the procedure described above. One benefit of the succession of cutting locations on the same blade such that at each location it is shaving off a thin layer of material, as shown in FIGS. 19 and 20, is that it greatly reduces the cutting force because thin shavings are free to curl out of the way of the advancing blade. Cutting a single thick plug of material would typically require a larger force, since a large volume of material would have to deform to let the knife through.

FIG. 21 shows two knife blades mounted on a single handle in axial view. One blade is a left handed helix, and the other blade is a right handed helix. A helical blade will generate a sideways force as it is going into the tissue. By having two helical blades curved in opposite directions, the sideways forces are opposite and cancel out. moreover, the knife would cut two grooves, which may be desirable in certain applications.

In one embodiment, the microknife is self-sharpening, where in one embodiment the knife can maintain its sharpness as the knife is used, where the knife's sharpness can be measured as a radius of curvature of the cutting edge. The microknife blade can be made to be self-sharpening by forming the knife of a thin layer of a relatively hard material (e.g., silicon nitride) and a support structure of a relatively soft material (e.g., silicon). When used to cut through a material, the softer support structure wears more quickly and exposes the harder material, which acts as the cutting edge of the knife. The sharpness of the microknife thus follows from the thickness of the harder material. For example, if the hard material is 100 Angstroms thick, the cutting edge will not be more than 100 Angstroms thick itself.

In an embodiment of a self-sharpening knife, the knife will automatically reach an equilibrium taper sloping up from the thin cutting edge to the thickness of the supporting body with continued use. Therefore, an initial slope of the cutting edge produced by etching during fabrication does not need to produce the final desired slope by itself. The etch may just approximate the desired shape, and then a mechanical abrasion process may be used to wear away the softer silicon and generate the final shape of the knife edge.

The different methods of forming the knife are a trade-off between the sophistication of the etch method to yield a desired slope, and the time spent on the abrasive wear-in process. At one end of the spectrum is the simple plasma etch with near vertical side walls, followed by a simple (but prolonged) abrasive wear-in to do 100% of the slope forming. At the other end of the spectrum is an anisotropic wet etch that produces the desired slope, and needing no abrasive wear-in. Where on this spectrum a process should fall depends on the details of the knife geometry that is needed for a given application, as well as on the facilities that are available for doing the fabrication.

Two basic strategies for generating the tapered slope from the cutting edge to the full thickness of the body of the knife include: (1) wet etching with an anisotropic etchant, and (2) deep reactive ion etching (using a plasma) with gray scale lithography to make the desired sloping sidewalls.

Wet etching produces straight edges in particular crystal directions. These edges are very precise as-etched, since they are defined by crystal planes. Plasma etching and gray scale lithography can produce any desired curved shape of knife edge, but the surface of the resulting knife blade tends to be relatively rough and irregular. In this case, etching can be followed by an abrasive process to make use of the self-sharpening property of the blade and achieve a smooth equilibrium slope to the blade edge.

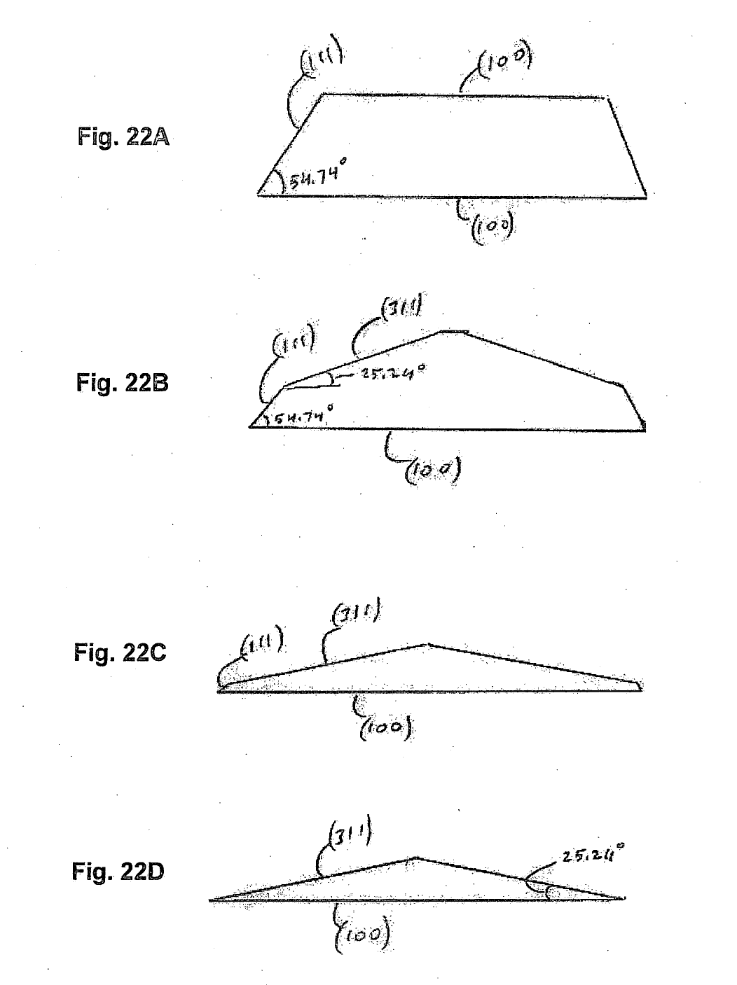

Wet etching may be used to make microknife blades for applications where a straight cutting edge is appropriate such as a microtome or a simple scalpel. Wet etching of single crystal silicon (100) wafer may make cutting edges that are formed by exposing (111) planes, or by exposing (311) planes. The angle of the slope at the blade edge formed by the intersection of a (111) plane with the masked (100) plane is 54.74 degrees. The angle formed by a (311) plane with the masked (100) plane is 25.2 degrees. FIGS. 22A through 22D show transverse cross sections through various cutting edge profiles that can be obtained by anisotropic wet etching of single crystal silicon oriented in the (100) direction. FIG. 22A shows the 54.7-degree slope that results when the (100) surface is protected by a masking layer, such as silicon dioxide. FIGS. 22B through 22D show the additional slope of 25.24 degrees that can be obtained by removing the mask layer from the (100) surface and then to continuing to etch further (i.e., maskless etching).

An abrasive process for achieving a desired blade geometry may comprise running the etched blade through an abrasive medium. Abrasive particles that may be used for silicon are cerium oxide, which may be in a slurry or imbedded in a polymer, a felt, or fabric polishing pad. The blade is preferably moved through the abrasive in the same orientation as it would be used in cutting tissue in surgery. Other abrasives, such as alumina, may be used in other embodiments. In addition, an oxidizer may be added, such as hydrogen peroxide, to speed up the formation of an oxide layer after fresh silicon is exposed by the abrasive. Moreover, a voltage may be applied (e.g., with the silicon as the anode) to further accelerate the silicon removal process. The forces applied to the microknife should generally be small. To keep the forces small, the microknife can be mounted on a low inertia compliant suspension as it is immersed in the moving abrasive.

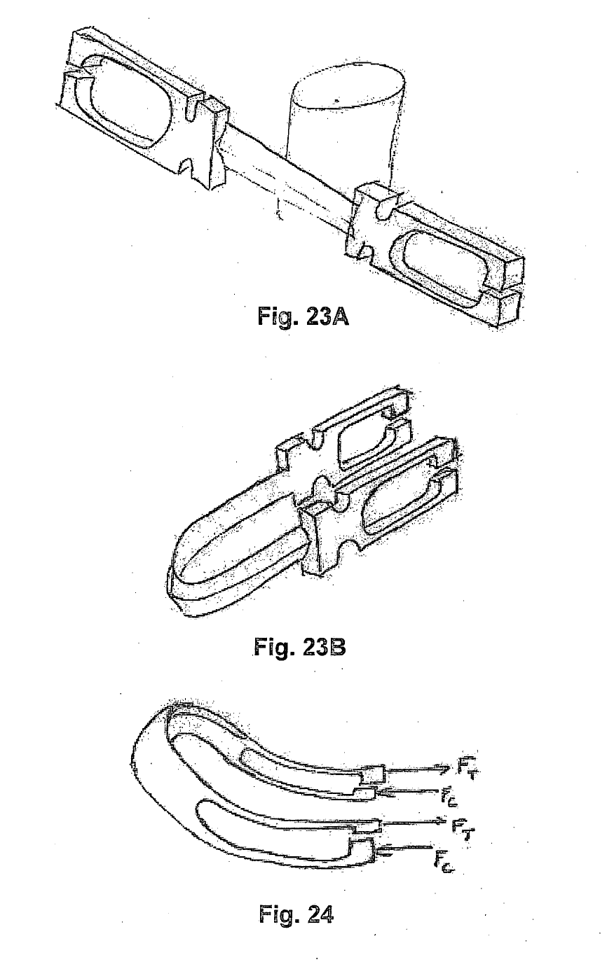

FIGS. 22A and 22B shows the formation of a curved blade that has been made using etching techniques to fabricate silicon structures. A process of hot plastic deformation, described above, is applied to form the desired curvature (in the example shown, a U-shaped blade, but other geometries such as helical geometries may be achieved as well). In this blade, supporting structures such as compliant flexures have been integrated with the blade, allowing the blade curved in one direction to then be deflected in another direction. FIG. 23 show the application of tensile forces (F_{T}) and compressive forces (F_{C}) to deflect the cutting edge out-of-plane, as the knife may be used to cut into a target tissue.

The planar microknife can be formed using any of a variety of known methods. In one embodiment, the microknife is formed by etching the blade 10 and cutting edge 20 structures from silicon, and possibly having a thin film of silicon nitride. For example, the following procedure is an example process in which the planar microknife may be formed, knife having supporting structures such as flexures integrated with the blade:
1. Begin with a SOI (silicon on insulator) wafer having desired device layer thickness for flexure beams (e.g., 50 to 100 microns).
2. Apply photoresist (PR) and pattern for flexures and areas that will have full device layer thickness (mask 1).
3. Perform an anisotropic deep trench plasma etch to BOX (buried oxide layer).
4. Clean the wafer.
5. Grow 1.5 microns thermal oxide.
6. Apply PR and pattern to expose oxide between knife blades (mask 2).
7. Apply 5% HF to thin exposed oxide to 1 micron.
8. Apply PR and pattern to expose oxide over knife blades (mask 3).
9. Apply 5% HF to thin exposed oxide over knife blades to 1 micron, and exposed oxide between knife blades to 0.5 micron.
10. Cover front side with PR.
11. Apply PR to back side and pattern to expose entire die at each die site (mask 4).
12. Put wafer in TMAH (25%, 80 C) to remove handle wafer silicon under each die site.
13. Apply 5% HF to remove exposed BOX from back side.
14. Clean the wafer.
15. Grow 100 angstrom thermal nitride on exposed silicon (bottom of device layer).
16. Apply 5% HF to front side to reduce front side oxide thickness by 0.5 micron to expose silicon in areas to be etched between knife blades.
17. Clean the wafer.
18. Etch exposed silicon in TMAH (e.g., 25%, 80 C) to form surfaces defined by (111) planes.
19. Apply 5% HF to front side to reduce front side oxide thickness by 0.5 micron to expose silicon of knife blade body.
20. Etch exposed silicon in TMAH to form surfaces defined by (311) planes.
21. Apply 5% HF to front side to remove all oxide.
22. Remove individual knives by breaking silicon tethers that connect them to the etched silicon frame.

Knife blades that are curved out-of-plane may be constructed using the following additional steps:
1. Set the blade in forming jig with blade against heated post (e.g., 1000° C.).
2. Move forming die to bend knife blade against heated post.
3. Let cool below 600° C.
4. Remove knife blade from forming jig.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure. The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. A three-dimensional cutting instrument comprising:
a blade; and
a cutting edge on at least one edge of the blade, the cutting edge offset at an angle from a cutting direction of the instrument,
wherein the radius of curvature of the cutting edge is less than 50 Angstroms, and
wherein the cutting edge and blade are curved in a direction having a vector component transverse to the cutting direction so that the blade does not lie entirely within a single plane.

2. The cutting instrument of claim 1, wherein the blade is formed from silicon.

3. The cutting instrument of claim 1, wherein the blade is U-shaped.

4. The cutting instrument of claim 1, wherein the blade is helical.

5. The cutting instrument of claim 4, wherein the helical blade is an elliptical helix.

6. The cutting instrument of claim 4, wherein the helical blade is a conical helix.

7. The cutting instrument of claim 1, wherein the blade forms a mirrored helix.

8. The cutting instrument of claim 1, further comprising:
a handle structure coupled to the blade.

9. The cutting instrument of claim 8, wherein the blade is helical and the handle structure comprises a rod, and the helical blade is mounted around the rod.

10. The cutting instrument of claim 8, wherein the handle structure and blade are configured to fit within a catheter.

11. The cutting instrument of claim 8, wherein the blade is mounted to the handle structure so that during operation of the instrument the blade is oriented with respect to tissue at an angle less than 45°.

12. The cutting instrument of claim 1, wherein the blade comprises a layer of cutting material and a layer of support material, the cutting material having a lower rate of wear than the support material, whereby the blade is self-sharpening due to the relative wear properties of the cutting and support materials.

13. A method for making a three-dimensional cutting instrument, the method comprising:
forming a planar blade, the blade having a cutting edge with a radius of curvature that is less than 50 Angstroms;
heating the planar blade;
plastically deforming the blade against a curved surface of a mandrel so that the cutting edge of the blade is curved in the direction of the deformation; and
cooling the blade.

14. The method of claim 13, wherein forming the planar blade comprises etching the blade in silicon.

15. The method of claim 13, wherein plastically deforming the blade produces a U-shaped blade.

16. The method of claim 13, wherein plastically deforming the blade produces a helical blade.

17. The method of claim 16, wherein the helical blade is an elliptical helix.

18. The method of claim 16, wherein the helical blade is a conical helix.

19. The method of claim 13, wherein plastically deforming the blade produces a blade in a mirrored helix geometry.

20. The method of claim 13, further comprising:
attaching the blade to a handle structure.

21. The method of claim 20, wherein the blade is helical and the handle structure comprises a rod, and the helical blade is mounted around the rod.

22. The method of claim 20, wherein the blade is attached to the handle structure so that during operation of the instrument the blade is oriented with respect to tissue at an angle less than 45°.

23. The method of claim 13, wherein the blade comprises a layer of cutting material and a layer of support material, the cutting material having a lower rate of wear than the support material, whereby the blade is self-sharpening due to the relative wear properties of the cutting and support materials.

24. The method of claim 13, wherein heating the planar blade comprises heating the mandrel and contacting the planar blade with the heated mandrel.

25. The method of claim 13, wherein the mandrel comprises a fused silica tube.

26. The method of claim 25, wherein heating the planar blade comprises passing an electrical current through a wire located inside the fused silica tube to heat the fused silica tube, and contacting the planar blade with the heated fused silica tube.

27. A method for performing microsurgery on tissue using a three-dimensional cutting instrument, the method comprising:
advancing a blade of the cutting instrument towards and into tissue, and has a cutting edge with a radius of curvature that is less than 50 Angstroms, and wherein the blade is curved in a direction having a vector component transverse to the direction that the blade is advanced into the tissue so that the blade does lie entirely within a single plane; and
separating a strip of the tissue with the blade.

28. The method of claim 27, wherein the blade comprises silicon.

29. The method of claim 27, wherein the blade is U-shaped.

30. The method of claim 27, wherein the blade is helical.

31. The method of claim 30, wherein the helical blade is an elliptical helix.

32. The method of claim 30, wherein the helical blade is a conical helix.

33. The method of claim 27, wherein the blade forms a mirrored helix.

34. The method of claim 27, wherein the blade is coupled to a handle structure, and advancing the blade of the cutting instrument is performed by applying a force to the handle structure.

35. The method of claim 34, wherein the blade is helical and the handle structure comprises a rod, and the helical blade is mounted around the rod.

36. The method of claim 34, wherein advancing the blade of the cutting instrument is performed while the blade contacts the tissue through an opening in a catheter.

37. The method of claim 34, wherein the blade comprises a layer of cutting material and a layer of support material, the cutting material having a rate of wear lower than the support material, whereby the blade is self-sharpening due to the relative wear properties of the cutting and support materials.

38. The method of claim 27, wherein the blade is oriented with respect to tissue at an angle less than 45°.

39. The method of claim 27, wherein the blade is helical and oriented with respect to the tissue at a relief angle in the range of 0 to 45 degrees and at a pitch angle in the range of 5 to 45 degrees.

40. The method of claim 27, further comprising:
advancing the blade through the tissue to remove the separated strip from the tissue.

41. The method of claim 27, further comprising:
ceasing the advancing of the blade through the tissue to leave the separated strip connected to the tissue.

42. A method for cutting a material, the method comprising:
providing a blade having a helical cutting edge; and
advancing the blade through a material so that the helical blade separates a strip of material, the advancing performed without substantially turning the helical edge in a direction of rotation that is parallel to the direction of the advancing.
